(19) United States
(12) Patent Application Publication
(10) Pub. No.: US 2005/0131369 A1
(43) Pub. Date: Jun. 16, 2005

(54) Sanitary napkin

(75) Inventor: Harvey Stanley Benson, Rockport, TX (US)

Correspondence Address: Richard R. Ruble;JACKSON WALKER L.L.P., Suite 2100, 112 E. Pecan Street, San Antonio, TX, 78205, US

(73) Assignee: LOTION PUFF, INC.

(21) Appl. No.: 10/733,590

(22) Filed: Dec. 11, 2003

(51) Int. Cl. A61F 13/15; A61F 13/20

(52) U.S. Cl. 604/385.01

(57) ABSTRACT

The present invention provides an absorbent article having a fluid pervious top sheet, a fluid impervious bottom sheet and an absorbent core therebetween. The absorbent article having a supplemental member defined by a slit cut through the top sheet and into the absorbent core. The supplemental member being capable of movement between a retracted position and a projected position wherein at least a portion of the supplemental member is located above the top sheet of the article. The supplemental member being capable of insertion into the body of the user such that the article may function both as a tampon and as a conventional pad. In one embodiment, the absorbent core of the present invention is capable of expansion and contraction depending upon the relative position of the supplemental member.

### FIELD OF THE INVENTION

The present invention relates generally to feminine hygiene articles and, more particularly, to a unique sanitary napkin.

### BACKGROUND OF THE INVENTION

Known sanitary napkin designs typically utilize an absorbent element having a body-facing side for receiving body fluids and an undergarment facing side which may be lined or treated to render it impervious to body fluids. Although these external sanitary napkins are calculated to have sufficient absorbent capacity to collect and contain menstrual discharge, they may fall short in protecting against leakage.

Known designs also comprise absorptive tampons designed for insertion into the body whereby, menstrual discharge is expected to be absorbed prior to exiting the user's body. However, during times of heavy menstrual flow, fluid may leak past the tampon such that an external pad may also be required. Further, some users may experience discomfort related to the use of a tampon, especially when the normal bulk of the tampon has been increased by absorption of fluids.

There remains a need for a sanitary napkin design capable of accommodating the individual requirements each user.

### SUMMARY OF THE INVENTION

Accordingly, the present invention provides an absorbent article having a fluid pervious top sheet, a fluid impervious bottom sheet, and an absorbent core therebetween. The absorbent article having a supplemental member defined by a slit cut through the top sheet and into the absorbent core.

The supplemental member of the present invention being hinged such that it is capable of movement between a retracted position and a projected position wherein at least a portion of the supplemental member is located above the top sheet of the article. When in a projected position, the supplemental member may be inserted into the body of the user such that the article functions both as a tampon and as a conventional pad. In one embodiment, at least a portion of the absorbent core of the present invention is capable of expanding and contracting depending upon the position of the supplemental member.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawing, wherein:

FIG. 1 is a perspective view of one embodiment of the present invention illustrating the supplemental member in a projected position.

FIG. 2 is a side, elevation, cross-sectional view of one embodiment of the present invention illustrating the supplemental member in a projected position.

FIG. 3 is a plan view of one embodiment of the present invention illustrating the supplemental member in a retracted position.

FIG. 4 is a side, elevation, cross-sectional view of one embodiment of the present invention.

### DETAILED DESCRIPTION OF THE INVENTION

The present invention is herein described as an absorbent article (10) placeable between the body and undergarments of a user.

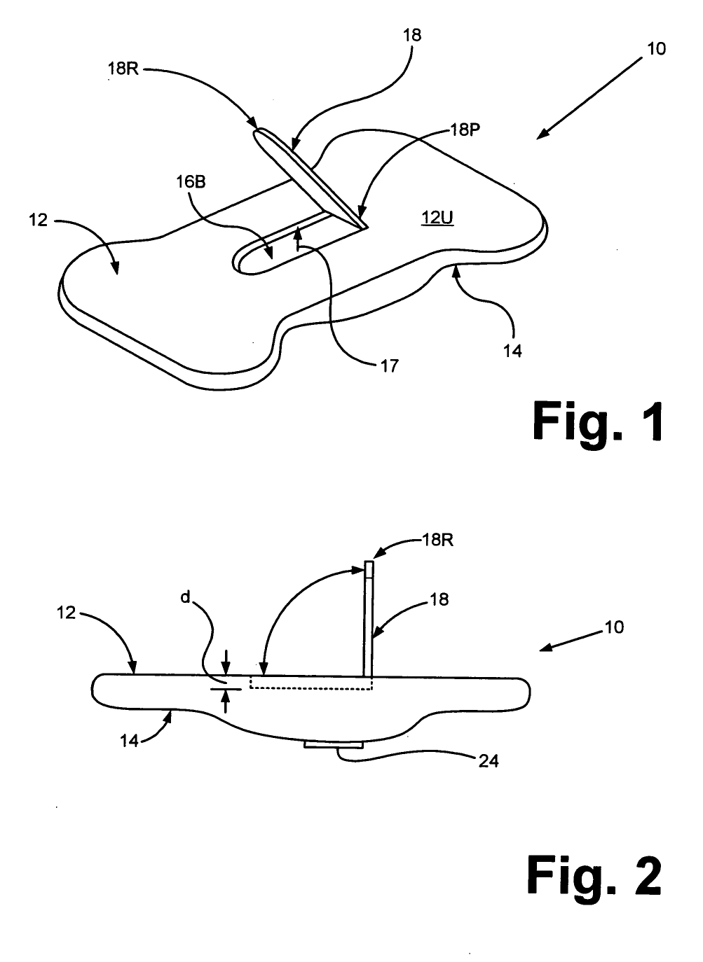

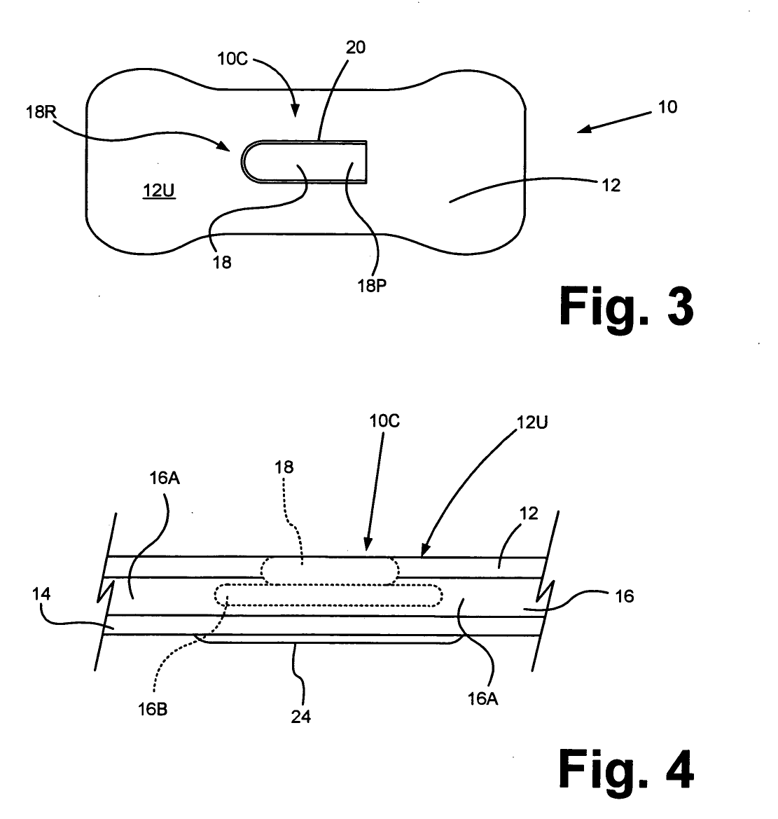

Referring to FIGS. 1-3, the absorbent article (10) of the present invention comprises a top sheet (12) having an upper surface (12U) for engaging at least a portion of the body of the user. In one embodiment, the top sheet (12) of the present invention is composed of a material through which fluids may pass. Thus, menstrual fluids are allowed to proceed through the fluid pervious top sheet such that they do not remain in continuous contact with the body of the user. In one embodiment, the absorbent article of the present invention has a generally elongated shape, such as an hourglass or elongated oval.

The present invention provides a bottom sheet (14) designed to protect the undergarments of the user. In one embodiment, the bottom sheet (14) may comprise a flexible material which may be a tear-resistant, non-woven, perforated plastic film or a combination thereof. Further, the bottom sheet may be composed of an elastic material, thus allowing the bottom sheet to bend and/or flex as needed. In one embodiment, the bottom sheet comprises a fluid impervious or fluid resistant material, such as polyethylene film, to prevent the passage of fluids onto the user's undergarments. In one embodiment, the bottom sheet (14) of the article (10) is equipped with an adhesive member (24) for removably attaching the article (10) to the undergarments of the user, if desired.

The absorbent article (10) of the present invention provides an absorbent core (16) designed to absorb menstrual flow and/or fluids. In one embodiment, the absorbent core (16) is located between the top sheet (12) and the bottom sheet (14) such that fluids passing through the top sheet (12) are thus absorbed. Any suitable material or combination of materials, such as are known in the art, may be utilized for the absorbent core (16).

In one embodiment, a central portion (10C) of the absorbent article (10) comprises a supplemental member (18). In one embodiment, the supplemental member of the present invention is defined by a slit (20) cut through the top sheet (12) of the article and into the absorbent core (16). The relative thickness of the supplemental member may be adjusted based upon the depth at which the slit is cut into the absorbent core (16).

The supplemental member (18) has a removed end (18R) and a proximate end (18P). In one embodiment, the proximate end (18P) is fixed to the top sheet (12) of the article (10) via a hinge-like attachment. Such hinge-like attachment being accomplished through a seam or other known attachment method.

The removed end (18R) of the supplemental member (18) is capable of movement between a retracted position and a projected position. The removed end (18R) being at least partly above the top sheet (12) of the article (10) when in a projected position. In one embodiment, the proximate end (18P) of the supplemental member (18) is located in a forward portion of the article (10) such that the member, when moved into a projected position, has a relatively large area of rearwardly facing pad available for absorption, should the need arise. In one embodiment, the length and/or shape of slit (20) allows the removed end (18R) to rise and flex in an upwardly direction while proximate end (18P) remains firmly attached to the article (10).

In one embodiment, the supplemental member (18) may be moved to the projected position for insertion into the body of the user. In this manner, the unique design of the present invention allows the article (10) to function both as a tampon-like absorption device and as an absorbent pad. This feature of the present invention is especially useful for those situations in which the user requires a measure of extra protection. Further, the use of the supplemental member (18) while in a projected position, acts as a stabilizer to hold the article (10) in place during movement/activity.

The supplemental member may utilize any number of geometric configurations. In one embodiment, the supplemental member (18) has a generally U-shaped configuration. In another embodiment, the supplemental member (18) has a substantially cylindrical configuration. It being understood that the geometric figuration of the supplemental member may be adjusted based upon the dimensions and orientation of the slit (20).

Referring to FIG. 4, the absorbent core (16) of the present invention is designed to work in concert with the supplemental member (18) regardless of its position. In short, the thickness, consistency, and/or composition of all or a portion of the absorbent core may be varied in order to provide the user with the most comfortable protection possible. In one embodiment, at least a portion of the absorbent core (16) comprises a material capable of compressing into the available space below the supplemental member while it is in a retracted position. In this embodiment, the material is also capable of expanding into the space left by the supplemental member (18) upon obtaining a projected position, as illustrated by arrow (17) of FIG. 1. This feature of the present invention allows the article (10) to maintain a level of linear integrity regardless of the relative position of the supplemental member (18).

In one embodiment, the absorbent core (16) comprises a first section (16A) and a second section (16B). In one embodiment, the second section (16B) comprises a material capable of expansion and/or contraction as described above. It being understood that any number of known materials may be utilized for this purpose and that the materials utilized for the first and second (16A and 16B, respectively) sections of the core (16) may be the same or may vary.

Although the invention has been described with reference to specific embodiments, this description is not meant to be construed in a limited sense. Various modifications of the disclosed embodiments, as well as alternative embodiments of the invention, will become apparent to persons skilled in the art upon reference to the description of the invention. It is, therefore, contemplated that the appended claims will cover such modifications that fall within the scope of the invention.

1. An absorbent article, said article being placeable between the body of a user and undergarments worn by the user, said article comprising:
a liquid pervious top sheet having an upper surface for engaging at least a portion of the body of the user;
a liquid impervious bottom sheet for protecting the undergarments of the user;
an absorbent core provided between said top sheet and said bottom sheet; and
a supplemental member defined by a slit cut through said upper surface of said top sheet and to a preselected depth into said absorbent core, said supplemental member having a removed end capable of movement between a retracted position and a projected position wherein said supplemental member extends at least partly above said top sheet of said article.

2. The article of claim 1, wherein said supplemental member is located in a central portion of said article.

3. The article of claim 1, further comprising an adhesive member for removeably attaching said bottom sheet to the undergarments of the user.

4. The article of claim 2, wherein said absorbent core comprises a first section and a second section, said second section located substantially beneath said supplemental member.

5. The article of claim 4, wherein said second section comprises an absorbent material capable of (a) compression when said supplemental member is in a retracted position and (b) expansion when said supplemental member is in a projected position.

6. The article of claim 1, wherein said supplemental member comprises a substantially cylindrical configuration.

7. The article of claim 1, wherein said supplemental member comprises a substantially u-shaped configuration.
